(19) United States
(12) Patent Application Publication
(10) Pub. No.: US 2015/0037790 A1
(43) Pub. Date: Feb. 5, 2015

(54) CYTOSINE VARIANT DETECTION

(71) Applicant: The University of Southampton, Southampton (GB)

(72) Inventors: Keith R. Fox, Southampton (GB); Tom Brown, Southampton (GB); Scott T. Kimber, Southampton (GB)

(73) Assignee: The University of Southampton, Southampton (GB)

(21) Appl. No.: 14/325,067

(22) Filed: Jul. 7, 2014

Related U.S. Application Data
(60) Provisional application No. 61/843,272, filed on Jul. 5, 2013.

(51) Int. Cl. C12Q 1/68

(52) U.S. Cl. CPC C12Q 1/6813
USPC 435/6.11

(57) ABSTRACT

This invention relates to methods for variant cytosine detection, and kits and probes for variant cytosine detection. In particular the variant cytosine detection is related to detection of methylated cytosine, hydroxymethylated cytosine, carboxycytosine and/or formylcytosine in nucleic acid.

This claims the benefit of U.S. Provisional Application No. 61/843,272, filed Jul. 5, 2013, which is incorporated herein by reference in its entirety.

This invention relates to methods for variant cytosine detection, and kits and probes for variant cytosine detection. In particular the variant cytosine detection is related to detection of methylated cytosine, hydroxymethylated cytosine, carboxycytosine and/or formylcytosine in nucleic acid.

DNA methylation is a biochemical process involving the addition of a methyl group to the cytosine or adenine DNA nucleotides. Cytosine methylation, especially at CpG sites, acts as an epigenetic marker which affects gene expression and regulation.

It is important to the study of epigenetics that a methylated cytosine site can be detected in any given DNA sequence. The most commonly used methods for detecting 5-methylcytosine are direct sequencing after treatment with bisulphite (Shapiro R, Braverman B, Louis J B, Servis R E (1973) Nucleic acid reactivity and conformation. II. J Biol Chem 248(11):4060-4064) or protection from cleavage by methylation sensitive restriction enzymes.

Treatment of DNA with bisulphite (known as bisulphite sequencing) converts cytosine residues to uracil, but leaves 5-methylcytosine residues unaffected. Thus, bisulphite treatment introduces specific changes in the DNA sequence that depend on the methylation status of individual cytosine residues. Single-nucleotide resolution of the methylation status of a segment of DNA is achievable. Analysis can be performed on the altered sequence to retrieve the information. However, treating DNA with bisulphite is time consuming and it is difficult to achieve complete conversion of all the cytosine residues in the sequence reaction. Furthermore, the bisulphite reaction leaves the DNA vulnerable to degradation. Sequencing the DNA to determine where cytosine residues have been converted to uracil is also time consuming and costly.

Methylation sensitive restriction enzymes are limited by the fact that they are highly specific to a given sequence of nucleic acid. Therefore, restriction enzymes cannot be used to query any given sequence of nucleic acid.

An aim of the present invention is to provide an improved method of detecting variant cytosine residues, such as methylated cytosines.

According to a first aspect of the invention, there is provided, a method for distinguishing between a variant and a non-variant cytosine residue in a nucleic acid sequence, comprising:
providing the nucleic acid in a double stranded format, wherein the cytosine residue is:
   (i) unpaired;
   (ii) paired with an abasic site;
   (iii) paired with a non-nucleosidic linker;
   (iv) paired with an unnatural nucleotide; or
   (v) mismatched; and
treating the nucleic acid with cytosine DNA glycosylase (CDG) to depyrimidate non-variant cytosine residues, wherein variant cytosine residues remain intact;
treating the nucleic acid in order to cut the nucleic acid strand at the site of any depyrimidated residue;
determining if the nucleic acid has been cut.

The nucleic acid may be provided in a double stranded format by annealing at least one probe oligo to the nucleic acid. The probe oligo may be complementary to upstream and downstream flanking sequences of a cytosine residue, and further comprising:
(i) the abasic site;
(ii) the non-nucleosidic linker;
(iii) the unnatural nucleotide, or
(iv) the mismatched residue,
at the residue position of the probe oligo that is opposing the cytosine residue.

The probe oligo may be arranged to be complementary to the nucleic acid upstream and downstream of the variant cytosine residue, but not complementary to the variant cytosine residue, such that the cytosine residue is arranged to be unpaired. Where the variant cytosine is unpaired, the cytosine may be arranged to be looped-out when the probe oligo is annealed to the nucleic acid. The nucleic acid may be arranged to form a loop when the probe oligo is annealed to the nucleic acid, wherein the loop comprises the cytosine residue. The cytosine residue may be unpaired by the probe oligo, thereby forcing the cytosine into a loop upon annealing/hybridisation of the probe oligo to the nucleic acid.

Looping-out the cytosine advantageously makes it available to the cytosine DNA glycosylase active site.

First and second probe oligos may be provided, wherein the first probe oligo is complementary to the sequence immediately downstream of the cytosine residue, and the second probe oligo is complementary to the sequence immediately upstream of the cytosine residue, such that a gap between the first and second probe oligos leaves the cytosine residue unpaired. The term “immediately upstream” or “immediately downstream” may be understood to be an adjacent residue to the cytosine, or one residue upstream/downstream of the cytosine residue.

Determining if the nucleic acid has been cut may comprise PCR amplifying the nucleic acid with a pair of primers complementary to sequences flanking the site of the cytosine residue. Variant cytosine residues will not be depyrimidated and cut, resulting in successful PCR amplification, thereby confirming the presence of a variant form of the cytosine residue. Non-variant cytosine residues will be depyrimidated and cut resulting in unsuccessful PCR amplification, thereby confirming the presence of a non-variant form of the cytosine residue.

Determining if the nucleic acid has been cut may comprise annealing a molecular beacon to the nucleic acid, wherein the molecular beacon is complementary to flanking regions upstream and downstream of the nucleic acid, and wherein the molecular beacon is arranged to signal a successful annealing to the nucleic acid. Molecular beacons are oligonucleotide hybridization probes that can report the presence of specific nucleic acids, for example in homogenous solutions. Molecular beacons may be hairpin shaped molecules with an internally quenched fluorophore whose fluorescence is restored when they bind to a target nucleic acid sequence. The molecular beacon may be a HyBeacon probe (HAIN Lifesciences). HyBeacon probes are single-stranded fluorescence labelled probes complementary to the nucleic acid. In unbound condition of the HyBeacon probes, the fluorophore can not emit fluorescence. After hybridization with the nucleic acid, excitation and measurement of fluorescence is possible.

The method of the invention advantageously provides an accurate residue specific method for detecting cytosine variation, such as methylation. The method is suitable for hemimethylated and fully methylated detection, and it can be used on any cytosine residue, where CpG sites are not required.

The variant cytosine may be selected from any of the group comprising methylated cytosine, hydroxymethylated cytosine, carboxylated cytosine, formylated cytosine and combinations thereof.

The prevalence of variant cytosine residues in a nucleic acid sample may be quantified. The PCR may be real time-PCR (RT-PCR). The PCR product may be detected and/or quantified by gel electrophoresis, HPLC, or fluorescence imaging. A molecular beacon probe, such as HyBeacon, may be used to detect and/or quantify the PCR product, or the cut/non-cut nucleic acid.

The nucleic acid may comprise variant cytosine residue(s) on only one of the two strands of a double stranded molecule, for example, the nucleic acid may be hemimethylated, where only one strand of double stranded nucleic acid is methylated. The nucleic acid may be hemimethylated, hemihydroxymethylated, hemicarboxylated and/or hemiformylated. Alternatively the nucleic acid may comprise variant cytosine residues on both complementary strands. For example, both strands of a double stranded nucleic acid may be methylated.

The nucleic acid may comprise DNA. The nucleic acid may be mammalian. The nucleic acid may be human. The nucleic acid may be genomic DNA, or a fragment thereof. The nucleic acid may be chromosomal DNA, or a fragment thereof.

The nucleic acid may be double stranded or single stranded. Where the nucleic acid is double stranded, the strands may be separated prior to annealing the probe oligo(s). For example, the double stranded nucleic acid may be heated above its melting temperature to separate the strands prior to annealing the probe oligo(s).

Annealing the probe oligo(s) may comprise mixing the probes with the nucleic acid sequence to be analysed under conditions suitable for sequence specific annealing of the probe oligo(s) to the complementary nucleic acid sequence. The skilled person will be capable of adjusting conditions, such as temperature and/or salt concentrations, to achieve specific annealing of the probe oligo(s).

The probe oligo may comprise DNA. The probe oligo may comprise nucleotide analogues, such as PNA, LNA (locked nucleic acid) or BNA (bridged nucleic acid). The probe oligo may comprise DNA and nucleotide analogues, such as PNA, LNA or BNA.

Where the probe oligo comprises both DNA and other nucleotide analogues, the nucleotide analogues may flank the DNA upstream and/or downstream. The probe oligo may comprise an abasic site. An abasic site may also be known as an AP site (apurinic/apyrimidinic site), and may be understood to be a location in DNA that has neither a purine nor a pyrimidine base.

The probe oligo may comprise a linker molecule. The probe oligo may comprise a non-nucleosidic linker residue. The non-nucleosidic linker may be a spacer molecule. The non-nucleosidic linker may be hexaethyl glycol. The non-nucleosidic linker may be propanediol or octanediol. The non-nucleosidic linker may be any natural or synthetic molecule capable of linking two strands of nucleic acid (for example 5′ to 3′ or 3′ to 5′). The linker may covalently link the strands of nucleic acid.

Using a linker, such as hexathyl glycol, has the benefit that it is not recognised by polymerases during PCR amplification. This may reduce the potential for artefacts that may arise from amplification of the probe oligo.

The probe oligo may comprise an unnatural nucleotide. The unnatural nucleotide may comprise a pyrene nucleotide, an anthraquinone analogue, or anthraquinone pyrrolidine.

Using an unnatural nucleotide, such as anthraquinone pyrrolidine, may provide the benefit of acting like a physical wedge, which pushes the cytosine residue of the nucleic acid out of the normal structural conformation of double stranded nucleic acid. This ensures that it is available for the active site of the cytosine DNA glycosylase.

The term “mismatch” may be understood to be the pairing of one residue to another residue, which do not naturally complement each other or form a pair. For example, the nucleotide residues of CG would be considered a matched pair, whereas CA, CT or CC pairings would be considered mismatched. The mismatch residue may be adenine at the site opposite the cytosine. The mismatch residue may be cytosine at the site opposite the cytosine. The mismatch residue may be thymine at the site opposite the cytosine. The cytosine residue of the nucleic may not be paired with guanine.

The probe oligo may be between about 7 and about 40 nucleotides in length, the probe oligo may be between about 10 and about 30 nucleotides in length, or between about 10 and about 20 nucleotides in length. The probe oligo may be between about 20 and about 40 nucleotides in length. It is understood that the abasic site, non-nucleosidic linker or unnatural nucleotide, may be counted as a single residue when determining the length of the probe.

Where at least two probe oligos are used for creating a gap opposite the cytosine, the first and/or second probe oligo may comprise DNA The first and/or second probe oligo may comprise nucleotide analogues, such as PNA, LNA or BNA. The first and/or second probe oligo may comprise DNA and nucleotide analogues, such as PNA, LNA or BNA. Where the first and/or second probe oligo comprises both DNA and other nucleotide analogues, the nucleotide analogues may not be located at the 5′ and/or the 3′ end of the probe oligo. The first and/or second probe oligo may be between about 5 and about 50 nucleotides in length, or between about 10 and about 40 nucleotides in length. The first and/or second probe oligo may be between about 15 and about 40 nucleotides in length.

The cytosine DNA glycosylase (CDG) may be a modified form of uracil DNA glycosylase (UDG). The cytosine DNA glycosylase (CDG) may be a modified form of the uracil DNA glycosylase (UDG) according to SEQ ID No. 1. The cytosine DNA glycosylase (CDG) may be a modified form of the uracil DNA glycosylase (UDG) according to SEQ ID No. 5. The cytosine DNA glycosylase (CDG) may be a modified form of the uracil DNA glycosylase (UDG) according to SEQ ID No. 6. The cytosine DNA glycosylase may be substantially as described in Kwon et al (2003) Chemistry & Biology. 10(4):351-9; and Kavli et al (1996) EMBO J. 15(13) 3442-7 incorporated herein by reference. The modification of the UDG may comprise a mutated active site.

The mutated active site of the UDG may comprise a L191A substitution and/or a N123D substitution, for example where the UDG is E. coli UDG. The mutated active site of the UDG may comprise a L272A substitution and/or a N204D substitution, for example where the UDG is human UDG. The mutated active site of the UDG may comprise a L281A substitution and/or a N213D substitution, for example where the UDG is human UDG. The CDG may be bacterial origin, mammalian origin, or human origin. The cytosine DNA glycosylase may be human origin. The cytosine DNA glycosylase may be E. coli origin. Where sequence variations exist between species and strains of UDG enzymes, it is understood that equivalent substitutions may be provided as determined by conserved sequence motifs. For example a pBLAST alignment between UDG enzymes of different strains or species will identify conserved residues, where one or more of the equivalent substitutions may be selected.

Where the UDG is human isoform 1 in accordance with SEQ ID NO. 5 herein, the mutated active site may comprise a L272A substitution and/or a N204D substitution.

Where the UDG is human isoform 2 in accordance with SEQ ID NO. 6 herein, the mutated active site may comprise a L281A substitution and/or a N213D substitution.

The CDG may comprise SEQ ID NO. 2, SEQ ID NO. 3, or SEQ ID NO. 4. The CDG may comprise SEQ ID NO. 4. The CDG may comprise a sequence having at least 80% identity with SEQ ID NO. 2, SEQ ID NO. 3, or SEQ ID NO. 4. The CDG may comprise a sequence having at least 90% identity with SEQ ID NO. 2, SEQ ID NO. 3, or SEQ ID NO. 4. The CDG may comprise a sequence having at least 95% identity with SEQ ID NO. 2, SEQ ID NO. 3, or SEQ ID NO. 4. The CDG may comprise a sequence having at least 98% identity with SEQ ID NO. 2, SEQ ID NO. 3, or SEQ ID NO. 4. The CDG may comprise a sequence having at least 99% identity with SEQ ID NO. 2, SEQ ID NO. 3, or SEQ ID NO. 4.

The CDG may comprise SEQ ID NO. 5 having substitutions comprising L272A, and/or N204D. The CDG may comprise a sequence having at least 80% identity with SEQ ID NO. 5 and having substitutions comprising L272A and/or N204D. The CDG may comprise a sequence having at least 90% identity with SEQ ID NO. 5 and having substitutions comprising L272A and/or N204D. The CDG may comprise a sequence having at least 95% identity with SEQ ID NO. 5 and having substitutions comprising L272A and/or N204D. The CDG may comprise a sequence having at least 98% identity with SEQ ID NO. 5 and having substitutions comprising L272A and/or N204D. The CDG may comprise a sequence having at least 99% identity with SEQ ID NO. 5 and having substitutions comprising L272A and/or N204D.

The CDG may comprise SEQ ID NO. 6 having substitutions comprising L281A and/or N213D. The CDG may comprise a sequence having at least 80% identity with SEQ ID NO. 6 and having substitutions comprising L281A and/or N213D. The CDG may comprise a sequence having at least 90% identity with SEQ ID NO. 6 and having substitutions comprising L281A and/or N213D. The CDG may comprise a sequence having at least 95% identity with SEQ ID NO. 6 and having substitutions comprising L281A and/or N213D. The CDG may comprise a sequence having at least 98% identity with SEQ ID NO. 6 and having substitutions comprising L281A and/or N213D. The CDG may comprise a sequence having at least 99% identity with SEQ ID NO. 6 and having substitutions comprising L281A and/or N213D.

The skilled person will understand that CDG enzyme variants comprising further mutations, elongation or truncation may be provided within the scope of this invention, where the CDG enzyme variants will retain the functional activity of cytosine depyrimidation.

Treating the probed nucleic acid with cytosine DNA glycosylase (CDG) may comprise incubating the nucleic acid with CDG for between about 1 hour and about 24 hours. Treating the probed nucleic acid with cytosine DNA glycosylase (CDG) may comprise incubating the nucleic acid with CDG for between about 1 hour and about 5 hours, or between about 2 hours and about 4 hours. Treating the probed nucleic acid with cytosine DNA glycosylase (CDG) may comprise incubating the nucleic acid with CDG for at least 1 hour. Treating the probed nucleic acid with cytosine DNA glycosylase (CDG) may comprise incubating the nucleic acid with CDG for at least 2 hours. Treating the probed nucleic acid with cytosine DNA glycosylase (CDG) may comprise incubating the nucleic acid with CDG for less than 24 hours, or less than 12 hours. Treating the probed nucleic acid with cytosine DNA glycosylase (CDG) may comprise incubating the nucleic acid with CDG for between about 2 hours and about 24 hours.

Treating the nucleic acid to cut the strand at sites of depyrimidation may be by an apurinic/apyrimidinic (AP) endonuclease, such as APE1, or heating in alkali. Treating the nucleic acid to cut the strand at sites of depyrimidation may be by heating the nucleic acid with piperidine or NaOH, such as about 10% piperidine, or about 0.1M NaOH. The heating may be carried out at between about 80° C. and about 100° C. The heating may be carried out at about 95° C.

Single stranded nucleic acid complementary to the nucleic acid sequence may be degraded, sequestered or removed prior to the PCR amplification. Single stranded or non-annealed nucleic acid may be degraded, sequestered or removed prior to the PCR amplification. Single stranded nucleic acid may be degraded by the action of the cytosine DNA glycosylase, which cuts single stranded nucleic acid. Single stranded nucleic acid may be degraded by a single strand nuclease after annealing the probe oligo; or after annealing the first and second probe oligos to the nucleic acid sequence. Single stranded nucleic acid complementary to the nucleic acid sequence may be removed by binding it to immobilised complementary oligonucleotides or tags.

A plurality of probe oligos, such as two or more probe oligos, may be used to query the cytosine variation status at multiple sites on the nucleic acid. Where a plurality of probe oligos are used, the reaction may be in a single reaction composition, or in multiple separate reaction compositions. A plurality of different probe oligos may be used in an array of reactions comprising the same or different nucleic acid sequences. A plurality of the same probe oligos may be used in an array of reactions comprising the same or different nucleic acid sequences. A plurality of the same probe oligos may be used in an array of reactions comprising the same nucleic acid sequences collected from different individuals, strains, or species or collected under different conditions, such as different growth conditions, or collected at different times.

Two or more different nucleic acid sequences may be analysed to detect variant cytosine residues in the same reaction, or in separate reactions on an array. Two or more of the same nucleic acid sequences isolated from different individual organisms may be analysed to detect variant cytosine residues in separate reactions on an array. Two or more of the same nucleic acid sequences isolated from the same organism may be analysed to detect variant cytosine residues in separate reactions on an array, wherein the same nucleic acid sequence may be isolated from the organism at different times or under different conditions. The array may be a microarray.

The method may not comprise the use of bisulphite and/or sequencing of the nucleic acid.

According to another aspect of the present invention, there is provided a cytosine DNA glycosylase for use to detect a variant cytosine residue in a nucleic acid sequence.

The use of the cytosine DNA glycosylase may be according to the method of the invention herein.

According to another aspect of the present invention, there is provided a kit for detecting a variant cytosine residue in a nucleic acid sequence, the kite comprising:
a cytosine DNA glycosylase; and/or
(a) a probe oligo comprising:
   (i) an abasic site
   (ii) a non-nucleosidic residue; or
   (iii) an unnatural nucleotide residue
   (iv) mismatched residue; or
(b) a first probe oligo arranged to be complementary to a first sequence of nucleic acid, and a second probe oligo arranged to be complementary to a second sequence of nucleic acid, wherein the first and second sequence of nucleic acid are on the same strand, and spaced apart by a single nucleic acid residue.

The kit may comprise primers for PCR amplification of the nucleic acid. The kit may comprise one or more molecular beacon probes.

According to another aspect of the present invention, there is provided a method for distinguishing between a variant and a non-variant cytosine residue in a nucleic acid sequence, comprising:
providing the nucleic acid in a double stranded format, wherein the cytosine residue is:
   (i) unpaired;
   (ii) paired with an abasic site;
   (iii) paired with a non-nucleosidic linker;
   (iv) paired with an unnatural nucleotide; or
   (v) mismatched; and
treating the nucleic acid with cytosine DNA glycosylase (CDG) to depyrimidate non-variant cytosine residues, wherein variant cytosine residues remain intact;
replicating the treated nucleic acid by a polymerase; and
detecting any change in nucleic acid sequence at the site of the variant cytosine residue.

The change in nucleic acid sequence may be effected by the polymerase as it reads through the depyrimidated non-variant cytosine residue.

The change in nucleic acid sequence may be detected by a molecular beacon, such as a HyBeacon probe. The molecular beacon may hybridise to a changed nucleic acid sequence at a different temperature relative to the unchanged nucleic acid sequence. The change in nucleic acid sequence may be detected by sequencing. The change in nucleic acid sequence may be detected by restriction digest. The change in nucleic acid sequence at the site of the variant cytosine residue may be a change to an adenine residue or a thymine residue.

Replicating the treated nucleic acid by a polymerase may comprise PCR amplification. The PCR amplification may be quantitative, such as RT-PCR amplification.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

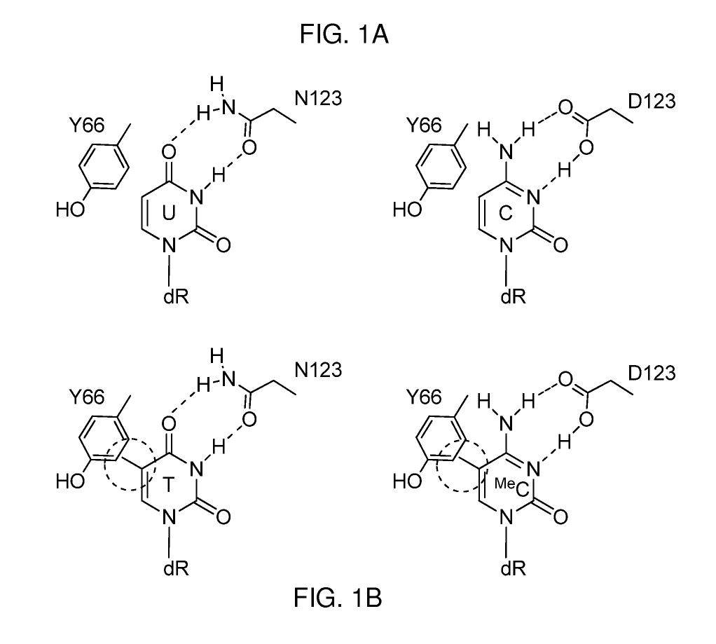

FIG. 1 illustrates the UDG enzyme and CDG enzyme mode of action. FIG. 1A shows interaction of U with N123 in uracil DNA glycosylase and proposed recognition of C by D123 in the N123D mutant. FIG. 1B shows exclusion of T and ^{Me}C caused by steric clash between their 5-methyl groups and Y66 (circled).

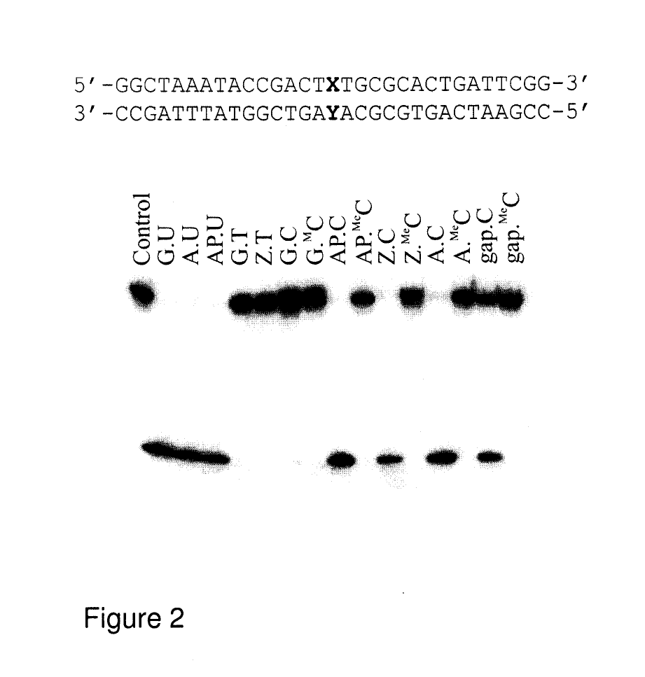

FIG. 2 shows CYDG cleavage of 31 mer fragments containing a central U, T, C or ^{Me}C opposite different bases. The ³²P labelled duplex substrates (˜50 nM) were incubated with ˜1.25 μM CYDG for 24 hours and then cleaved by boiling in 10% piperidine. The products were resolved on a 12.5% denaturing polyacrylamide gel.

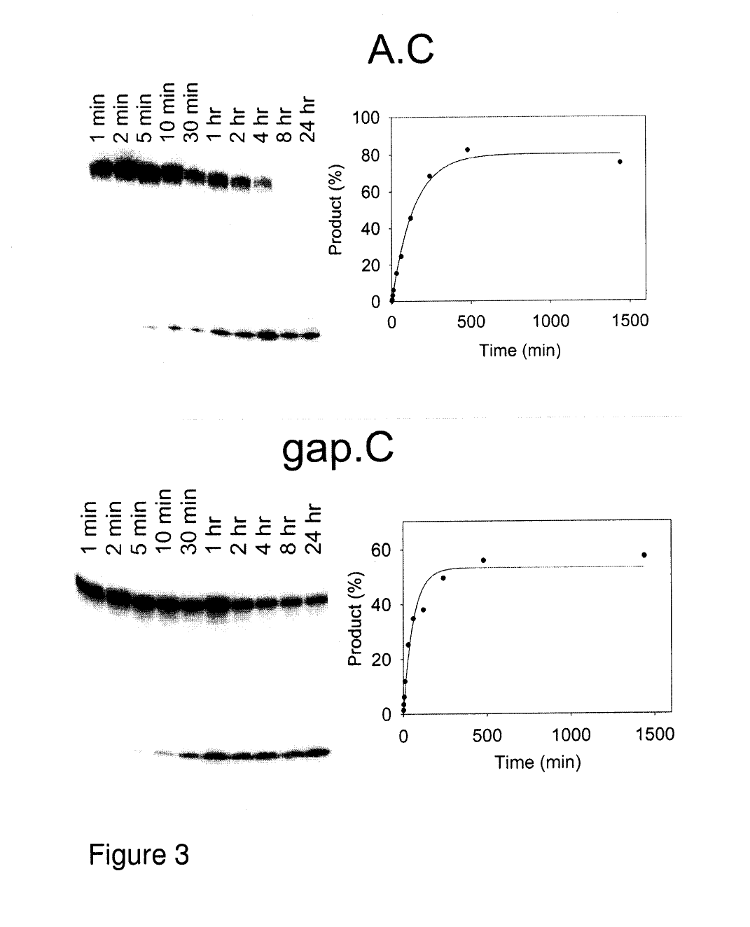

FIG. 3 provides representative gels showing the kinetics of cleavage of A.C and gap.C by CYDG. The products were resolved on a 12.5% denaturing polyacrylamide gel after boiling in 10% (v/v) piperidine.

It has been determined whether CYDG can discriminate between C and ^{Me}C, in the same way that UDG discriminates between U and T (FIG. 1). The cleavage selectivity of CYDG is determined and it has been shown to remove cytosine, but not methylcytosine, when it is mispaired with A or opposite an abasic site.

### Methods

### Preparation of Enzymes.

The sequence of E. coli UDG was cloned between the EcoRI and HindIII sites of pUC18. Site-directed mutagenesis generated the L191A mutation, which was followed by the N123D mutation. The sequence was then subcloned into pET28a and inserted between the EcoRI and NdeI sites The enzyme was expressed in BL21(DE3)pLysS cells, which were induced with 0.2 mM IPTG for three hours. The cells were lysed by sonication, purified using a Ni-NTA (His Trap FF Crude; GE Healthcare) and eluted in 250 mM imidazole. The enzyme was concentrated and further purified using a 20 mL 10000 MW Vivaspin column (Fisher Scientific).

### Preparation of Oligonucleotides.

Oligonucleotides were synthesized on an Applied Biosystems ABI 394 automated DNA/RNA synthesizer on the 0.2 or 1 μM scale using standard methods. Phosphoramidite monomers and other reagents were purchased from Applied Biosystems or Link Technologies. The pyrrolidine anthraquinone phosphoramidite was purchased from Berry & Associates. Each 31 mer oligonucleotide was radiolabelled at its 5′-end with γ-³²P[ATP] using T4 polynucleotide kinase (New England Biolabs), purified by denaturing PAGE, and resuspended in 10 mM MES pH 6.3 containing 25 mM NaCl and 2.5 mM MgCl₂). These were mixed with an excess of the unlabelled complementary oligonucleotides and annealed by slowly cooling from 95° C. to 4° C.

### Enzyme Cleavage.

Radiolabelled DNA (approximately 50 nM) was incubated with CYDG (typically 1.25 μM) for up to 24 h, removing samples from the reaction mixture at various time intervals. The reaction was stopped using 10% piperidine (v/v) and boiled at 95° C. for 20 min to cleave the phosphodiester backbone. The samples were lyophilised, resuspended in 5 μL loading buffer (80% (v/v) formamide, 10 mM EDTA, 10 mM NaOH and 0.1% (w/v) bromophenol blue) and run on a 12.5% denaturing polyacrylamide gel containing 8 M urea. The gel was then fixed, dried, subjected to phosphorimaging and analysed using ImageQuantTL. Experiments were performed in triplicate and k_{cat} values were determined using SigmaPlot by fitting to a single exponential rise to maximum to plots of percent cleaved against time. The rate of cleavage of some substrates was very low (less than 10% cleaved after 24 hours incubation). In these instances an estimate of the rate constant was obtained from the fraction cleaved at a given time, assuming a simple exponential process.

### Results

### Generation of CYDG (N123D, L191A)

Initial attempts to prepare the N123D mutant of E. coli UDG, which should have CDG activity, were unsuccessful, confirming that this enzyme is cytotoxic in E. coli (14, 15). Indeed we were unable to construct this mutant, even when the sequence was cloned within the polylinker of pUC19. The L191A mutant was therefore first introduced into UDG (generating UYDG), which was followed by the second N123D mutation to produce CYDG. The mutations were generated in pUC18 and then subcloned into pET28a followed by expression of the protein in E. coli.

### Excision Properties of CYDG

The activity and specificity of CYDG were tested against a range of double and single stranded DNA templates. Synthetic 31 mer oligonucleotide substrates were designed so as to pair U, T, C or ^{Me}C with G, A, AP (abasic site), Z (anthraquinone pyrrolidine) or a gap using two 15 mer oligonucleotides (Table 1).

TABLE 1
Oligonucleotides used to generate the sub-
strates A.C(G), G:C, gap.C, Long gap.C,
ssC(polyA) and ssC(GAT) to characterise the
cleavage rates of CYDG. Target base shown in
bold and underlined.

| Substrate | Sequence |
|---|---|
| A.C | 5′-CCGAATCAGTGCGCACAGTCGGTATTTAGCC-3′ |
| | 3′-GGCTTAGTCACGCGTATCAGCCATAAATCGG-5′ |
| A.C(G) | 5′-CCGAATCAGTGCGCGCGGTCGGTATTTAGCC-3′ |
| | 3′-GGCTTAGTCACGCGCACCAGCCATAAATCGG-5′ |
| G.C | 5′-CGAATAATTATATAACATATATATATTTAGC-3′ |
| | 3′-GCTTATTAATATATTGTATATATATAAATCG-3′ |
| gap.C | 5′-CCGAATCAGTGCGCACAGTCGGTATTTAGCC-3′ |
| | 3′-GGCTTAGTCACGCGT TCAGCCATAAATCGG-5′ |
| Long gap.C | 5′-CCGTACTGAATCAGTGCGCACAGTCGGTATT |
| | TACGATAGCC-3′ |
| | 3′-GGCATGACTTAGTCACGCGT TCAGCCATAA |
| | ATGCTATCGG-5′ |
| ssC(polyA) | 5′-AAAAAAAAAAAAAAACAAAAAAAAAAAAAAA-3′ |
| SSC(GAT) | 5′-GGATAAATAGGGAGTCTGAGAAGTGATTAGG-3′ |

Previous studies have used a pyrene nucleoside (7, 8, 15) as a plug to force the base into the active site; we used anthraquinone pyrrolidine as a similar bulky nucleotide analogue. The results, after incubating all the substrates with an excess of the enzyme, are shown in FIG. 2. CYDG cleaves all the sequences with a central cytosine, except when it is paired with guanine. In contrast none of the sequences with a central methylcytosine are cleaved, confirming that the 5-methyl group of cytosine is excluded from the active site, in a similar fashion to exclusion of the 5-methyl group of T.

As expected, cleavage is observed when C is opposed with the bulky anthraquinone analogue, as previously observed with a pyrene nucleotide (15). More surprisingly, cleavage is also observed when C is placed opposite any other base, except G. C is cleaved when positioned opposite A, an abasic site or a gap. This suggests that L191 is not required to “push” the cytosine into the active site if it is not involved in a stable base pair. L191 may have a more important role in base “plugging” rather than “pushing” (9). CYDG has residual activity against uracil, even when this is positioned opposite adenine, but showed no activity towards thymine in any base pair combination.

Determination of k_{cat}

The kinetics of cleavage of C by CYDG where examined when it is placed opposite various bases. Representative cleavage profiles are shown in FIG. 3 and the data is summarised in Table 2.

TABLE 2
k_{cat} values for CYDG cleavage of different DNA substrates. No
cleavage was observed for any substrate containing methylcytosine.

| Substrate | k_{cat} (min⁻¹) | Rel |
|---|---|---|
| A.C | 0.006 ± 0.001 | 1.7 |
| A.C(G)¹ | 0.0001 | ~0.02 |
| AP.C | 0.014 ± 0.003 | 4.0 |
| Z.C | 0.10 ± 0.02 | 29 |
| ssC(polyA)¹ | 0.0003 ± 0.0001 | ~0.07 |
| ssC(GAT)¹ | 0.0001 | ~0.02 |
| G.C | ND | <0.001 |
| gap.C² | 0.016 ± 0.002 | 4.6 |
| Long gap.C | 0.0072 ± 0.0007 | 2.0 |
| G.U | 0.36 ± 0.04 | 100 |
| A.U | 0.020 ± 0.004 | 5.6 |

ND—no cleavage detected after 24 hours. Values represent the average of three independent determinations.
¹k_{cat} values were estimated from single time points at 24 hrs A.C(G), 60 mins ssC(polyA) and 4 hrs ssC(GAT).
²gap.C k_{cat} only 50% of the substrate was cleaved. Rel indicates the cleavage rate relative to that of GU (100).

Reaction with the substrate containing a single AC mismatch produced a single product at a rate of 0.006±0.001 min⁻¹. The presence of a single product confirms that the enzyme does not cleave C when paired with G since this fragment contains several GC base pairs. The excision of uracil from GU (0.36±0.04 min⁻¹) is approximately 60-fold faster, but the observation that cleavage at AU is about 20-fold slower than GU (0.0.020±0.04 min⁻¹) suggests that the enzyme is best able to cleave C or U when in they are in an unstable (non-Watson-Crick) base pair. Anthraquinone pyrrolidine was included opposite C so as to force the target base into an extrahelical conformation. This produced the fastest cleavage rate at C (0.10±0.02 min⁻¹), faster even than AU, though again no reaction is observed at Z. ^{Me}C. These results suggest that base pair stability plays a major role in determining the rate of cleavage. This is further confirmed by experiments with the sequence in which the AC mismatch is flanked by GC base pairs [A.C(G)] for which cleavage is reduced by about 100-fold compared to AC flanked by AT base pairs. Fast cleavage was also achieved with gap.C (0.016±0.002 min⁻¹), which contains a gap opposite the C residue, allowing the unpaired cytosine to enter the active site of CYDG more easily. However, only 50% of this substrate was cleaved (FIG. 2B), while all other substrates were completely digested. This difference is probably due to the lower Tₘ of the duplexes formed by these split oligos, which is close to the reaction temperature. We therefore examined cleavage of an extended DNA substrate that contained an additional five base pairs on either side of the central C (long gap.C). The extent of cleavage was improved to 80% with this longer substrate, though the reaction proceeded at a slightly slower rate. The lower cleavage efficiency may also be because CYDG binds with high affinity to the gap on the opposite strand, consistent with the observation that UDG has high affinity for AP sites protecting them from further mutagenesis during base excision repair (13).

The ability of CYDG to cleave Cs in a single stranded DNA substrate was examined. Two substrates containing a single cytosine were used for these experiments;

ssC(polyA) contains a single C residue within a polydA tract, while ssC(GAT) contains a single C within a mixed sequence of G, A and T. Although UDG cuts single-stranded Us faster than those paired with A or G (17), only slow cleavage of both single-stranded DNAs by CYDG was observed.

### Discussion

### Discrimination Between C and ^{Me}C

CYDG, derived from E. coli UDG, was shown to be able to discriminate between cytosine and 5-methylcytosine. No activity against ^{Me}C was detected in any of the substrates tested, while C is efficiently cleaved, except when paired with G. In UDG Y66 is positioned close to the 5 position of the pyrimidine base and the 5-methyl group is sterically excluded. Alteration of the hydrogen bonding pattern at N123 changes the base selectivity, but the mutant enzyme is still able to discriminate between pyrimidine and 5-methylpyrimidine. A similar effect with human CDG is observed, though this enzyme has weak activity against C when paired with G. The lack of activity of CYDG against GC base pairs presents the possibility of using this enzyme to probe the methylation status of a specific cytosine, by mispairing it with another base such as adenine.

### Excision Properties

CYDG cleaves cytosine when it is unpaired or mispaired, and the stability of the base pair determines the rate of cleavage (18, 19). CYDG excised cytosine from Z.C faster than uracil from A.U, presumably because the mispaired cytosine is more easily forced into an extrahelical configuration than uracil in the Watson-Crick AU pair. The faster cleavage of gap.C and AP.C occurs because there is no base opposite the C. If GC base pairs flank the target cytosine then the rate of cleavage at AC is dramatically reduced as a result of the increased local DNA stability (20) and the inability of CYDG to flip the base into the active site. CYDG retains uracil DNA glycosylase activity despite the N123D mutation since free rotation of the aspartate side chain can still present the correct hydrogen bonding pattern for interacting with U (21). Although the activity of CYDG is greatly reduced compared with wild type UDG, its catalytic activity is similar to that of many other DNA glycosylases (22-25).

### The Role of L191.

The ability of CYDG to excise uracil from AU but not cytosine from GC suggests that the major role of L191 is to plug the space left after base flipping, rather than actively assisting the mechanism of base flipping itself (9). The binding of CYDG to the duplex and the distortion it causes to the DNA (9, 13, 26) appears to be sufficient to destabilise an AU but not GC base pairs.

### Conclusions

It is shown that CYDG is able to discriminate between cytosine and 5-methylcytosine. Cytosine-DNA glycosylase activity is observed when C is unpaired or in an unstable (non Watson-Crick) base pair, while no activity is observed at ^{Me}C in any base pair combination.

### Enzyme Sequences

Sequence of E. coli Uracil DNA Glycosylase (UDG)

(SEQ ID NO. 1)

MANELTWHDVLAEEKQQPYFLNTLQTVASERQSGVTIYPPQKDVFNAFRF
TELGDVKVVILGQDPYHGPGQAHGLAFSVRPGIAIPPSLLNMYKELENTI
PGFTRPNHGYLESWARQGVLLLNTVLTVRAGQAHSHASLGWETFTDKVIS
LINQHREGVVFLLWGSHAQKKGAIIDKQRHHVLKAPHPSPLSAHRGFFGC
NHFVLANQWLEQRGETPIDWMPVLPAESE
Sequence of L191A mutant (UYDG)

(SEQ ID NO. 2)

MANELTWHDVLAEEKQQPYFLNTLQTVASERQSGVTIYPPQKDVFNAFRF
TELGDVKVVILGQDPYHGPGQAHGLAFSVRPGIAIPPSLLNMYKELENTI
PGFTRPNHGYLESWARQGVLLLNTVLTVRAGQAHSHASLGWETFTDKVIS
LINQHREGVVFLLWGSHAQKKGAIIDKQRHHVLKAPHPSPASAHRGFFGC
NHFVLANQWLEQRGETPIDWMPVLPAESE
Sequence of N123D mutant

(SEQ ID NO. 3)

MANELTWHDVLAEEKQQPYFLNTLQTVASERQSGVTIYPPQKDVFNAFRF
TELGDVKVVILGQDPYHGPGQAHGLAFSVRPGIAIPPSLLNMYKELENTI
PGFTRPNHGYLESWARQGVLLLDTVLTVRAGQAHSHASLGWETFTDKVIS
LINQHREGVVFLLWGSHAQKKGAIIDKQRHHVLKAPHPSPLSAHRGFFGC
NHFVLANQWLEQRGETPIDWMPVLPAESE
Sequence of L191A, N123D double mutant (CYDG)

(SEQ ID NO. 4)

MANELTWHDVLAEEKQQPYFLNTLQTVASERQSGVTIYPPQKDVFNAFRF
TELGDVKVVILGQDPYHGPGQAHGLAFSVRPGIAIPPSLLNMYKELENTI
PGFTRPNHGYLESWARQGVLLLDTVLTVRAGQAHSHASLGWETFTDKVIS
LINQHREGVVFLLWGSHAQKKGAIIDKQRHHVLKAPHPSPASAHRGFFGC
NHFVLANQWLEQRGETPIDWMPVLPAESE
Sequence of human UDG isoform 1

(SEQ ID NO. 5)

MIGQKTLYSFFSPSPARKRHAPSPEPAVQGTGVAGVPEESGDAAAIPAKK
APAGQEEPGTPPSSPLSAEQLDRIQRNKAAALLRLAARNVPVGFGESWKK
HLSGEFGKPYFIKLMGFVAEERKHYTVYPPPHQVFTWTQMCDIKDVKVVI
LGQDPYHGPNQAHGLCFSVQRPVPPPPSLENIYKELSTDIEDFVHPGHGD
LSGWAKQGVLLLNAVLTVRAHQANSHKERGWEQFTDAVVSWLNQNSNGLV
FLLWGSYAQKKGSAIDRKRHHVLQTAHPSPLSVYRGFFGCRHFSKTNELL
QKSGKKPIDWKEL
Sequence of human UDG isoform 2

(SEQ ID NO. 6)

MGVFCLGPWGLGRKLRTPGKGPLQLLSRLCGDHLQAIPAKKAPAGQEEPG
TPPSSPLSAEQLDRIQRNKAAALLRLAARNVPVGFGESWKKHLSGEFGKP
YFIKLMGFVAEERKHYTVYPPPHQVFTWTQMCDIKDVKVVILGQDPYHGP
NQAHGLCFSVQRPVPPPPSLENIYKELSTDIEDFVHPGHGDLSGWAKQGV
LLLNAVLTVRAHQANSHKERGWEQFTDAVVSWLNQNSNGLVFLLWGSYAQ
KKGSAIDRKRHHVLQTAHPSPLSVYRGFFGCRHFSKTNELLQKSGKKPID
WKEL

### REFERENCES

1. Lindahl T, Nyberg B (1974) Heat-induced deamination of cytosine residues in deoxyribonucleic acid. Biochemistry 13(16):3405-3410.
2. Lindahl T (1974) An N-glycosidase from Escherichia coli that releases free uracil from DNA containing deaminated cytosine residues. Proc Natl Acad Sci USA 71(9):3649-3653.
3. Tye B K, Nyman P O, Lehman I R, Hochhauser S, Weiss B (1977) Transient accumulation of Okazaki fragments as a result of uracil incorporation into nascent DNA. Proc Natl Acad Sci USA 74(1):154-157.
4. Stivers J T, Pankiewicz K W, Watanabe K A (1999) Kinetic mechanism of damage site recognition and uracil flipping by Escherichia coli uracil DNA glycosylase. Biochemistry 38:952-963.
5. Savva R, McAuley-Hecht K, Brown T, Pearl L H (1995) The structural basis of specific base-excision repair by uracil-DNA glycosylase. Nature 373:487-493.
6. Mol C D, et al. (1995) Crystal structure and mutational analysis of human uracil-DNA glycosylase: structural basis for specificity and catalysis. Cell 80:869-878.
7. Jiang Y L, Kwon K, Stivers J T (2001) Turning on uracil-DNA glycosylase using a pyrene nucleotide switch. J Biol Chem 276(45):42347-42354.
8. Jiang Y L, Stivers J T (2002) Base-flipping mutations of uracil DNA glycosylase: substrate rescue using a pyrene nucleotide wedge. Biochemistry 41:11248-11254.
9. Jiang Y L, Stivers J T (2002) Mutational analysis of the base-flipping mechanism of uracil DNA glycosylase. Biochemistry 41:11236-11247.
10. Handa P, Acharya N, Varshney U (2002) Effects of mutations at tyrosine 66 and asparagine 123 in the active site pocket of Escherichia coli uracil DNA glycosylase on uracil excision from synthetic DNA oligomers: evidence for the occurrence of long-range interactions between the enzyme and substrate. Nucleic Acids Res 30(14):3086-3095.
11. Drohat A C, Stivers J T (2000) Escherichia coli uracil DNA glycosylase: NMR characterization of the short hydrogen bond from His 187 to uracil O2. Biochemistry 39:11865-11875.
12. Drohat A C, et al. (1999) Heteronuclear NMR and crystallographic studies of wild-type and H187Q Escherichia coli uracil DNA glycosylase: electrophilic catalysis of uracil expulsion by a neutral histidine 187. Biochemistry 38:11876-11886.
13. Parikh S S, et al. (1998) Base excision repair initiation revealed by crystal structures and binding kinetics of human uracil-DNA glycosylase with DNA. EMBO J. 17:5214-5226.
14. Kavli B, et al. (1996) Excision of cytosine and thymine from DNA by mutants of human uracil-DNA glycosylase. EMBO J. 15(13):3442-3447.
15. Kwon K, Jiang Y L, Stivers J T (2003) Rational engineering of a DNA glycosylase specific for an unnatural cytosine:pyrene base pair. Chemistry & Biology 10:351-359.
16. Shapiro R, Braverman B, Louis J B, Servis R E (1973) Nucleic acid reactivity and conformation. II. Reaction of cytosine and uracil with sodium bisulfite. J Biol Chem 248(11):4060-4064.
17. Panayotou G, Brown T, Barlow T, Pearl L H, Savva R (1998) Direct measurement of the substrate preference of uracil-DNA glycosylase. J Biol Chem 273(1):45-50.
18. Krosky D J, Song F, Stivers J T (2005) The origins of high-affinity enzyme binding to an extrahelical DNA base. Biochemistry 44(16):5949-5959.
19. Krosky D J, Schwarz F P, Stivers J T (2004) Linear free energy correlations for enzymatic base flipping: how do damaged base pairs facilitate specific recognition? Biochemistry 43(14):4188-4195.
20. Seibert E, Ross J B, Osman R (2002) Role of DNA flexibility in sequence-dependent activity of uracil DNA glycosylase. Biochemistry 41(36):10976-10984.
21. Pearl L H (2000) Structure and function in the uracil-DNA glycosylase superfamily. Mutation Research 460:165-181.
22. Roy R, Brooks C, Mitra S (1994) Purification and biochemical characterization of recombinant N-methylpurine-DNA glycosylase of the mouse. Biochemistry 33(50):15131-15140.
23. Neddermann P, Jiricny J (1994) Efficient removal of uracil from G:U mispairs by the mismatch-specific thymine DNA glycosylase from HeLa cells. Proc Natl Acad Sci USA 91:1642-1646.
24. Bjelland S, Birkeland N K, Benneche T, Volden G, Seeberg E (1994) DNA glycosylase activities for thymine residues oxidized in the methyl group are functions of the AlkA enzyme in Escherichia coli. J Biol Chem 269(48):30489-30495.
25. Boiteux S, O'Connor T R, Lederer F, Gouyette A, Laval J (1990) Homogeneous Escherichia coli FPG protein. A DNA glycosylase which excises imidazole ring-opened purines and nicks DNA at apurinic/apyrimidinic sites. J Biol Chem 265(7):3916-3922.
26. Werner R M, et al. (2000) Stressing-out DNA? The contribution of serine-phosphodiester interactions in catalysis by uracil DNA glycosylase. Biochemistry 39:12585-12594.

1. A method for distinguishing between a variant and a non-variant cytosine residue in a nucleic acid sequence, comprising:
providing the nucleic acid in a double stranded format, wherein the cytosine residue is:
(i) unpaired;
(ii) paired with an abasic site;
(iii) paired with a non-nucleosidic linker;
(iv) paired with an unnatural nucleotide; or
(v) mismatched; and
treating the nucleic acid with cytosine DNA glycosylase (CDG) to depyrimidate non-variant cytosine residues, wherein variant cytosine residues remain intact;
treating the nucleic acid in order to cut the nucleic acid strand at the site of any depyrimidated residue;
determining if the nucleic acid has been cut.

2. The method of claim 1, wherein the nucleic acid is provided in a double stranded format by annealing at least one probe oligo to the nucleic acid.

3. The method of claim 2, wherein the probe oligo is complementary to upstream and downstream flanking sequences of a cytosine residue, and further comprising
(i) the abasic site;
(ii) the non-nucleosidic linker;
(iii) the unnatural nucleotide, or
(iv) the mismatched residue,
at the residue position of the probe oligo that is opposing the cytosine residue.

4. The method of claim 2, wherein first and second probe oligos are provided, wherein the first probe oligo is complementary to the sequence immediately downstream of the cytosine residue, and the second probe oligo is complementary to the sequence immediately upstream of the cytosine residue, such that a gap between the first and second probe oligos leaves the cytosine residue unpaired.

5. The method according to claim 1, wherein determining if the nucleic acid has been cut comprises PCR amplifying the nucleic acid with a pair of primers complementary to sequences flanking the site of the cytosine residue,
wherein variant cytosine residues will not be depyrimidated and cut, resulting in successful PCR amplification, thereby confirming the presence of a variant form of the cytosine residue; and
wherein non-variant cytosine residues will be depyrimidated and cut resulting in unsuccessful PCR amplification, thereby confirming the presence of a non-variant form of the cytosine residue.

6. (canceled)

7. The method according to claim 1, wherein determining if the nucleic acid has been cut comprises annealing a molecular beacon to the nucleic acid, wherein the molecular beacon is complementary to flanking regions upstream and downstream of the nucleic acid, and wherein the molecular beacon is arranged to signal a successful annealing to the nucleic acid.

8-11. (canceled)

12. The method according to claim 1, wherein the cytosine DNA glycosylase (CDG) is a modified form of uracil DNA glycosylase (UDG); and optionally, wherein the modification of the UDG comprises a mutated active site.

13. The method according to claim 12, wherein the mutated active site of the UDG comprises a L191A substitution and/or a N123D substitution; or
wherein the mutated active site of the UDG comprises a L272A substitution and/or a N204D substitution; or
wherein the mutated active site of the UDG comprises a L281A substitution and/or a N213D substitution; or
equivalent substitutions thereof where the same residue substitution is provided at an equivalent conserved residue having a different residue position.

14. The method according to claim 1, wherein the CDG comprises a sequence of at least 80% identity to any of the sequences selected from the group comprising SEQ ID NO. 2; SEQ ID NO. 3; SEQ ID NO. 4; SEQ ID NO. 5 having substitutions comprising L272A and/or N204D; and SEQ ID NO. 6 having substitutions comprising L281A and/or N213D.

15-18. (canceled)

19. The method according to claim 1, wherein two or more different nucleic acid sequences are analysed to detect variant cytosine residues in the same reaction, or in separate reactions on an array.

20-21. (canceled)

22. A method for distinguishing between a variant and a non-variant cytosine residue in a nucleic acid sequence, comprising:
providing the nucleic acid in a double stranded format, wherein the cytosine residue is:
(i) unpaired;
(ii) paired with an abasic site;
(iii) paired with a non-nucleosidic linker;
(iv) paired with an unnatural nucleotide; or
(v) mismatched; and
treating the nucleic acid with cytosine DNA glycosylase (CDG) to depyrimidate non-variant cytosine residues, wherein variant cytosine residues remain intact;
replicating the treated nucleic acid by a polymerase; and
detecting any change in nucleic acid sequence at the site of the variant cytosine residue.

23. The method according to claim 22, wherein the change in nucleic acid sequence is effected by the polymerase as it reads through the depyrimidated non-variant cytosine residue.

24. The method according to claim 22, wherein the change in nucleic acid sequence is detected by a molecular beacon probe.

25-26. (canceled)

27. The method according to claim 22, wherein the nucleic acid is provided in a double stranded format by annealing at least one probe oligo to the nucleic acid.

28. The method according to claim 27, wherein the probe oligo is complementary to upstream and downstream flanking sequences of a cytosine residue, and further comprising
(i) the abasic site;
(ii) the non-nucleosidic linker;
(iii) the unnatural nucleotide, or
(iv) the mismatched residue,
at the residue position of the probe oligo that is opposing the cytosine residue.

29. The method according to claim 27, wherein first and second probe oligos are provided, wherein the first probe oligo is complementary to the sequence immediately downstream of the cytosine residue, and the second probe oligo is complementary to the sequence immediately upstream of the cytosine residue, such that a gap between the first and second probe oligos leaves the cytosine residue unpaired.

30-33. (canceled)

34. The method according to claim 22, wherein the cytosine DNA glycosylase (CDG) is a modified form of uracil DNA glycosylase (UDG); and optionally, wherein the modification of the UDG comprises a mutated active site.

35. The method according to claim 34, wherein the mutated active site of the UDG comprises a L191A substitution and/or a N123D substitution; or
wherein the mutated active site of the UDG comprises a L272A substitution and/or a N204D substitution; or
wherein the mutated active site of the UDG comprises a L281A substitution and/or a N213D substitution; or
equivalent substitutions thereof where the same residue substitution is provided at an equivalent conserved residue having a different residue position.

36. The method according to claim 22, wherein the CDG comprises a sequence of at least 80% identity to any of the sequences selected from the group comprising SEQ ID NO. 2; SEQ ID NO. 3; SEQ ID NO. 4; SEQ ID NO. 5 having substitutions comprising L272A and/or N204D; and SEQ ID NO. 6 having substitutions comprising L281A and/or N213D.

37-42. (canceled)

43. A kit for detecting a variant cytosine residue in a nucleic acid sequence, the kit comprising:
a cytosine DNA glycosylase; and/or
(a) a probe oligo comprising:
(i) an abasic site
(ii) a non-nucleosidic residue; or
(iii) an unnatural nucleotide residue
(iv) mismatch residue; or
(b) a first probe oligo arranged to be complementary to a first sequence of nucleic acid, and a second probe oligo arranged to be complementary to a second sequence of nucleic acid, wherein the first and second sequence of nucleic acid are on the same strand, and spaced apart by a single nucleic acid residue.

44-48. (canceled)


SEQUENCE LISTING (23 sequences)

SEQ ID NO: 1 - protein, 229 aa, Escherichia coli
MANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESE

SEQ ID NO: 2 - protein, 229 aa, Escherichia coli
MANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPASAHRGFFGCNHFVLANQWLEQRGETPIDWMPVLPAESE

SEQ ID NO: 3 - protein, 229 aa, Escherichia coli
MANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLDTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESE

SEQ ID NO: 4 - protein, 229 aa, Escherichia coli
MANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLDTVLTVRAGQAHSHASLGWETFTDKVISLINQHREGVVFLLWGSHAQKKGAIIDKQRHHVLKAPHPSPASAHRGFFGCNHFVLANQWLEQRGETPIDWMPVLPAESE

SEQ ID NO: 5 - protein, 313 aa, Homo sapiens
MIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 6 - protein, 304 aa, Homo sapiens
MGVFCLGPWGLGRKLRTPGKGPLQLLSRLCGDHLQAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 7 - DNA, 31 bp, Artificial Sequence
  Primer
ccgaatcagtgcgcacagtcggtatttagcc

SEQ ID NO: 8 - DNA, 31 bp, Artificial Sequence
  Primer
ggcttagtcacgcgtatcagccataaatcgg

SEQ ID NO: 9 - DNA, 31 bp, Artificial Sequence
  Primer
ccgaatcagtgcgcgcggtcggtatttagcc

SEQ ID NO: 10 - DNA, 31 bp, Artificial Sequence
  Primer
ggcttagtcacgcgcaccagccataaatcgg

SEQ ID NO: 11 - DNA, 31 bp, Artificial Sequence
  Primer
cgaataattatataacatatatatatttagc

SEQ ID NO: 12 - DNA, 31 bp, Artificial Sequence
  Primer
gcttattaatatattgtatatatataaatcg

SEQ ID NO: 13 - DNA, 31 bp, Artificial Sequence; identical to SEQ ID NO: 7
  Primer

SEQ ID NO: 14 - DNA, 15 bp, Artificial Sequence
  Primer
ggcttagtcacgcgt

SEQ ID NO: 15 - DNA, 15 bp, Artificial Sequence
  Primer
tcagccataaatcgg

SEQ ID NO: 16 - DNA, 41 bp, Artificial Sequence
  Primer
ccgtactgaatcagtgcgcacagtcggtatttacgatagcc

SEQ ID NO: 17 - DNA, 20 bp, Artificial Sequence
  Primer
ggcatgacttagtcacgcgt

SEQ ID NO: 18 - DNA, 20 bp, Artificial Sequence
  Primer
tcagccataaatgctatcgg

SEQ ID NO: 19 - DNA, 31 bp, Artificial Sequence
  Primer
aaaaaaaaaaaaaaacaaaaaaaaaaaaaaa

SEQ ID NO: 20 - DNA, 31 bp, Artificial Sequence
  Primer
ggataaatagggagtctgagaagtgattagg

SEQ ID NO: 21 - DNA, 31 bp, Artificial Sequence
  Primer
ggctaaataccgactntgcgcactgattcgg

SEQ ID NO: 22 - DNA, 31 bp, Artificial Sequence
  Primer
ccgaatcagtgcgcanagtcggtatttagcc

SEQ ID NO: 23 - DNA, 30 bp, Artificial Sequence
  Primer
ggctaaataccgacttgcgcactgattcgg